(19) United States
(12) Patent Application Publication
(10) Pub. No.: US 2006/0018884 A1
(43) Pub. Date: Jan. 26, 2006

(54) Method for treating human tumor cells with a newcastle disease virus strain and a chemotherapeutic agent

(75) Inventors: Laszlo K. Csatary, Alexandria, VA (US); Joseph Szeberenyi, Sziget (HU); Zsolt Fabian, Sziget (HU)

Correspondence Address: LAW OFFICES OF STUART J. FRIEDMAN, 28930 RIDGE ROAD, MT. AIRY, MD, 21771, US

(73) Assignee: United Cancer Research Institute, Alexandria, VA (US)

(21) Appl. No.: 10/995,614

(22) Filed: Nov. 23, 2004

Related U.S. Application Data
(60) Provisional application No. 60/524,726, filed on Nov. 25, 2003.

(51) Int. Cl. A61K 35/76; A61K 38/16; A61K 31/525; A61K 33/24

(52) U.S. Cl. 424/93.6; 424/649; 514/251; 514/283; 514/8

(57) ABSTRACT

A method for treating human tumor cells to induce apoptotic cell death thereof includes the step of infecting the tumor cells with a combination of the Herefordshire strain of Newcastle Disease Virus and a chemotherapeutic agent. The range of concentrations of chemotherapeutic agent/Herefordshire strain is in the range of 100/1 to 1/1. Illustrative chemotherapeutic agents include cisplatin, methotrexate, vincristine, bleomycin and dacarbazine.

### CROSS-REFERENCE TO RELATED APPLICATIONS

This is a non-provisional application based upon U.S. provisional application Ser. No. 60/524,726, filed Nov. 25, 2003, now pending.

### FIELD OF THE INVENTION

The present invention relates to a method for treating human tumor cells to induce apoptotic cell death thereof with a Newcastle Disease Virus (NDV) strain and, more particularly, to a method for treating human tumor cells with a combination of a Newcastle Disease Virus strain and a chemotherapeutic agent.

### BACKGROUND OF THE INVENTION

It has already been demonstrated that the viral vaccine known as MTH-68/H, developed by United Cancer Research Institute (Ft. Lauderdale, Fla.) and available from UCRI Hungary Ltd. of Budapest, Hungary, containing highly purified, attenuated, mesogenic Herefordshire Newcastle Disease virus strain (hereinafter “Herefordshire strain”), has significant oncolytic capacity. The strain is nonpathogenic in humans and was found to have antineoplastic effects in patients with certain therapy resistant tumors, such as glioblastoma, colorectal cancer, melanoma and hematological malignancies. This oncolytic effect is, at least in part, due to its direct cytotoxicity. Cell death caused by this strain of Newcastle Disease Virus comes in the form of apoptosis. As used herein, the vaccine designation “MTH-68/H” refers to the aforementioned viral vaccine containing highly purified, attenuated Herefordshire strain.

Notwithstanding the acknowledged oncolytic effect of this Newcastle Disease viral strain it is believed that it can be a still more effective therapeutic agent against human tumor cells when used in combination with other oncolytic agents and that the combination will demonstrate a synergistic cytotoxicity which is more effective than either agent alone

### SUMMARY OF THE INVENTION

It is, therefore, a primary object of the present invention to characterize the oncolytic capacity of a purified, attenuated Herefordshire strain.

It is also an object of the present invention to demonstrate the effect of the Herefordshire strain on cell lines originating from human tumors.

It is another object of the present invention to demonstrate the cytotoxic effect of the Herefordshire strain in combination with chemotherapeutic agents in cell lines originating from human tumors.

The foregoing and other objects are achieved in accordance with the present invention by providing a method for treating human tumor cells to induce apoptotic cell death thereof comprising the step of infecting the tumor cells with the Herefordshire strain.

In another aspect of the present invention there is provided another method for treating human tumor cells to induce apoptotic cell death thereof comprising the steps of infecting the tumor cells with a combination of the Herefordshire strain and a chemotherapeutic agent.

In still another aspect of the present invention, the chemotherapeutic agents which evidence a synergistic cytotoxic effect, in combination with Herefordshire strain, on human tumor cells include: cisplatin, methotrexate, vincristine, bleomycin and dacarbazine.

In yet another aspect of the present invention, the ratio of chemotherapeutic agent to Herefordshire strain in the combination is in the range of 100:1 to 1:1.

### BRIEF DESCRIPTION OF THE DRAWINGS

FIG. 1 is a graphical representation of the cytotoxicity of MTH-68/H on control cells.

FIG. 2 is a graphical representation of the cytotoxicity of MTH-68/H on melanoma cell lines.

FIG. 3 is a graphical representation of the cytotoxicity of MTH-68/H on human colorectal cancer cell lines.

FIG. 4 is a graphical representation of the cytotoxicity of MTH-68/H on human prostate cancer cell lines.

FIG. 5 is a graphical representation of the cytotoxicity of MTH-68/H on human pancreas cancer cell lines.

FIG. 6 is a graphical representation of the cytotoxicity of MTH-68/H on human lung cancer cells.

FIG. 7 is a graphical representation of the cytotoxicity of MTH-68/H on human astrocytoma cells.

FIG. 8 is a graphical representation of the cytotoxicity of MTH-68/H on human A431 cancer cells.

FIG. 9 is a graphical representation of various NDV preparations on PANC-1 cells.

FIG. 10 is a graphical representation of various NDV preparations on HeLa cells.

FIG. 11 is a graphical representation of the cytotoxicity of the MTH-68/H/cisplatin combination on NCI-H460 cells.

FIG. 12 is a graphical representation of the cytotoxicity of the MTH-68/H/methotrexate combination on NCI-H460 cells.

FIG. 13 is a graphical representation of the cytotoxicity of the MTH-68/H/bleomycin combination on NCI-H460 cells.

FIG. 14 is a graphical representation of the cytotoxicity of the MTH-68/H/vincristine combination on HCT-116 cells.

FIG. 15 is a graphical representation of the cytotoxicity of the MTH-68/H/bleomycin combination on HCT-116 cells.

FIG. 16 is a graphical representation of the cytotoxicity of the MTH-68/H/dacarbazine combination on PC-3 cells.

FIG. 17 is a graphical representation of the cytotoxicity of the MTH-68/H/bleomycin combination on HeLa cells.

FIG. 18 is a graphical representation of the cytotoxicity of the MTH-68/H/bleomycin combination on HT-29 cells.

FIG. 19 is a graphical representation of the cytotoxicity of the MTH-68/H/chlorpromazine combination on PC-12 cells.

### DESCRIPTION OF THE PREFERRED EMBODIMENT

To demonstrate the cytotoxicity of the Herefordshire strain and the synergistic cytoxicity of combination of the Herefordshire strain with chemotherapeutic agents, several studies were conducted on various human cell lines. The main features of the cell lines used in these studies are summarized in Table I. The cell lines were cultured in media described in Table I. Cultures were infected with freshly suspended batches of virus preparations.

The following Newcastle disease virus strains were utilized:

Herefordshire Strain

The H (Herefordshire) strain of Newcastle Disease Virus was used in the form of the vaccine product MTH-68/H, obtained from UCRI Hungary Limited. The titre of the vaccine was 10^{8.3} EID in one ml. The vaccine was stored at −20° C. and protected from light. The lyophilized vaccine was dissolved in 1 ml sterile saline immediately prior to use.

LaSota

LaSota is an avirulent (lentogenic) ND vaccine virus strain. The titre of the vaccine was approximately 10⁹-10¹⁰ particles/ml. The vaccine was stored at −80° C.

Vitayest

Vitapest is an avirulent lentogenic ND vaccine virus strain. The titre of the vaccine was approximately 10⁹ particles/ml. The vaccine was stored at −80° C.

The following procedures were employed:

Cell Proliferation Assay

Proliferation and viability of cell lines under various experimental conditions

TABLE I
Cell lines used in this study

| | | | | | |
|---|---|---|---|---|---|
| | Species | | | | |
| | of | | | | |
| Cell line | origin | Tissue of origin | Comment | Culture medium | Source |

Non-cancerous cell lines

| | | | | | |
|---|---|---|---|---|---|
| NIH 3T3 | mouse | normal fibroblast | — | DMEM, | ATCC |
| | | | | 10% calf serum | |
| Rat-1 | rat | normal fibroblast | — | DMEM, | ATCC |
| | | | | 10% calf serum | |
| CHO | hamster | ovarian cells | — | DMEM, | from J. Szekeres |
| | | | | 20% FBS | |
| | human | foreskin fibroblast | primary culture | DMEM, | from G. Sáfrány |
| | | | | 20% FBS | |

Cancer cell lines

| | | | | | |
|---|---|---|---|---|---|
| PC12 | rat | phaeochromocytoma | — | DMEM, | from G. M. |
| | | | | 10% horse serum, | Cooper |
| | | | | 5% FBS | |
| PC12- | rat | phaeochromocytoma | expresses | DMEM, | from M. Pap |
| dn-p53 | | | dominant | 10% horse serum, | |
| | | | negative p53 | 5% FBS | |
| PC12- | rat | phaeochromocytoma | overexpresses | DMEM, | from Zs. Fábián |
| p53⁺ | | | wt-p53 | 10% horse serum, | |
| | | | | 5% FBS | |
| HeLa | human | cervix | low p53 | DMEM, | ATCC |
| | | adenocarcinoma | expression | 10% FBS | |
| MCF-7 | human | breast | p53-positive | DMEM, | ATCC |
| | | adenocarcinoma | | 10% FBS | |
| 293T | human | kidney | transformed | DMEM, | ATCC |
| | | | with adenovirus | 10% FBS | |
| | | | 5 DNA | | |
| Cos-7 | African | kidney | SV40- | DMEM, | ATCC |
| | green | | transformed | 10% FBS | |
| | monkey | | | | |
| PANC-1 | human | pancreas epitheloid | | RPMI1640 | from Schering |
| | | carcinoma | | 10% FBS | |
| | | | | supplemented | |
| | | | | with non-essential | |
| | | | | amiono acids and | |
| | | | | Na-pyruvate | |
| DU 145 | human | prostate carcinoma | brain metastasis | DMEM Ham′F12 | from Schering |
| | | | | 10% FBS | |
| NCI- | human | large cell lung cancer | positive for c- | DMEM Ham′F12 | from Schering |
| H460 | | | myb, v-fes, v- | 10% FBS | |
| | | | fms, c-raf 1, Ha- | | |
| | | | ras, Ki-ras and | | |
| | | | N-ras mRNA | | |
| HT-29 | human | colorectal cancer | p53 mutation, | DMEM Ham′F12 | from Schering |
| | | | truncated c-Met | 10% FBS | |
| PC-3 | human | prostate | bone metastasis | DMEM Ham′F12 | from Schering |
| | | adenocarcinoma | | 10% FBS | |
| B16 | mouse | melanoma | | DMEM, | from J. Szekeres |
| | | | | 10% FBS | |
| HCT-116 | human | colorectal cancer | activated ras | RPMI1640 | from Schering |
| | | | | 5% FBS | |
| U373 | human | astrocytoma | | DMEM, | from G. Sáfrány |
| | | | | 10% FBS | |
| HT-25 | human | colorectal cancer | | DMEM Ham′F12 | from J. Timár |
| | | | | 10% FBS | |
| HT-199 | human | melanoma | truncated c-Met | DMEM Ham′F12 | from J. Timár |
| | | | | 10% FBS | |
| WM983B | human | melanoma | truncated c-Met | DMEM Ham′F12 | from J. Timár |
| | | | | 10% FBS | |
| HT-168- | human | melanoma | truncated c-Met | DMEM Ham′F12 | from J. Timár |
| M1 | | | | 10% FBS | |
| A431 | human | epithelial cancer | HPV⁺ | DMEM Ham′F12 | from J. Timár |
| | | | low p53 | 5% FBS | |

were analyzed using the WST-1 kit of Roche Molecular Biochemicals following the manufacturers instructions. Optimal cell culture and assay conditions were determined in preliminary experiments. 1-4×10⁴ cells/well were seeded in standard culture medium in 24-well plates. Cultures were infected with the virus preparations at different titres (ranging from 100/1 to 1/100 cell/particle ratios) for 72 hours. WST-1 assays were performed for 120 minutes and light absorption (A₄₄₀) of media were taken in 96-well plates using an ELISA reader.

No-treatment and anisomycin-treated (1 μg/ml) cultures were used for negative and ctytotoxicity-positive controls, respectively.

Analysis of Virus Replication

Cells were cultured in 1 ml standard medium (see Table I) at a density of 4×10⁴ cells/well in 24-well dishes. Cells were infected with MTH-68/H, La Sota or Vitapest NDV strains at various cell/particle ratios. Incubations were performed for 72 hours, media were harvested and stored at −80° C. until titration. No treatment and anisomycin (1 μg/ml) treatment were used as controls.

Detection of DNA Fragmentation

2-5×10⁶ cells were cultured in DMEM (Dulbecco's modified Eagle medium) containing serum for 24 hours. Treatments were carried out as indicated in the legends of each of the Figures. Four positive control samples were incubated for 24 hours in serum-free DMEM or with anisomycin (1 μg/ml); for negative control they were kept in high-serum DMEM. After incubation for the time periods indicated in the Figures, cells were collected by scraping them into their own medium and then centrifuged at 1000 rpm for 5 minutes. The soluble DNA of these cells was extracted by the following method. Collected cells were solubilized on ice in extraction solution containing 0.5% Triton X-100, 5 mM TRIS pH 7.4, 5 mM EDTA for 20 minutes. Soluble DNA in the supernatant rsulting from centrifugation at 13500 rpm for 20 minutes at 4° C. was extracted with phenol/chloroform, chloroform, and finally precipitated with ethanol. The precipitates were treated with DNase free RNase A (Sigma-Aldrich, Steinheim, Germany (2 mg/ml) at 37° C. for 1 hour. DNA fragments were separated by electrophoresis in 1.8% agarose gels, and visualized on a UV transilluminator after staining the gel with SYBR Gold (Molecular Probes, Eugene, Oreg.).

Western Blot Analysis

Immunoblot analysis using antibodies against proteins indicated was performed as described by the manufacturers Cell Signaling (Beverly, Mass.) and Transduction Labs.

Protein concentrations were determined using the Bio-Rad Protein DC assay, and equivalent amounts of protein were resolved by SDS polyacrylamide gel electrophoresis using either 12% or 16% polyacrylamide gel. The proteins were transferred to an ECL membrane (Amersham Pharmacia Biotech AB., Uppsala, Sweden). Immune complexes were visualized using an enhanced chemiluminescence detection kit (Amersham Pharmacia Biotech AB) following the manufacturer's instructions. The following antibodies were used: Cleaved Caspase-3 (Rat specific), Cleaved Caspase-9 (Rat specific) from Cell Signaling (Beverly, Mass.) and PK R from Transduction Labs.

Electrophoretic Mobility Shift Assay (EMSA)

Nuclear extracts were prepared as described by Xu & Cooper in “Identification of a candidate c-mos repressor that restricts transcription of germ cell-specific genes”; Mol Cell Biol 1995; 15: 5369-5375. All subsequent steps were performed at 4° C. Cell pellets were washed twice in ice cold phosphate-buffered saline (1× PBS) and resuspended in 10 volumes of buffer containing 10 mM HEPES pH 7.9, 1.5 mM MgCl₂, 10 mM KCl, 0.5 mM dithiothreitol (DTT), protease inhibitors (Complete, Mini EDTA-free tablets, Boehringer Mannheim), phosphatase inhibitors (Phosphatase Inhibitor Cocktail, Sigma) and placed on ice for 10 minutes. After vigorous vortexing, nuclei were collected by centrifugation in a microcentrifuge and resuspended in 2 volumes of buffer containing 20 mM HEPES pH 7.9,25% glycerol, 420 mM NaCl, 1.5 mM MgCl₂, 0.2 mM EDTA, 0.5 mM DTT, protease inhibitors, phosphatase inhibitors and placed on ice for 20 minutes. After centrifugation in a microcentrifuge, the supernatants were saved, aliquoted and stored at −80° C. Protein concentrations were determined with the Bio-Rad Protein Assay Kit (Coomassie Brilliant Blue dye).

5′-end labeling of oligonucleotides was performed using [γ-³²P]-ATP and T4 polynucleotide kinase (Amersham Pharmacia Biotech Inc.) according to the manufacturer's protocol. After reconstitution of Ready-To-Go T4 polynucleotide kinase by adding 25 μl water and incubation at room termperature for 2-5 minutes, 5-10 pmol of 5′-ends of oligonucleotide, 22 μl water and 2 μl of [γ-³²P]-ATP (3000 Ci/mmol, 10 μCI/μl) were added, mixed gently and incubated at 37° C. for 30 minutes. The reaction was stopped by adding 5 μl of 250 mM EDTA. Labelled oligonucleotides were collected by Spin Column 10 (Sigma).

The protein-DNA binding reaction was performed as follows: 10-20 μg nuclear proteins were mixed with 1 μg poly(dI-dC), 100 ng nonspecific single-stranded oligonucleotide and 4 μl buffer containing 10 mM HEPES pH 7.5, 10% glycerol, 1 mM EDTA, 100 mM NaCl. Sufficient amount of distilled water was added to bring the reaction volume to 18 μl. After 15 minutes incubation at room temperature the mixture was completed with 2 μl, approximately 100 000 cpm of ³²P-labelled oligonucleotide (total reaction volume was 20 μl) and incubation at room temperature was continued for another 30 minutes.

DNA-protein complexes were electrophoresed in 5% non-denaturing polyacrylamide gel (5 ml 30% acrylamide-bisacrylamide mixture, 2.5 ml 10× Tris Base, Borate, EDTA buffer pH 8.3, 17.5 ml distilled water, 20 μl TEMED, 50 μl 25% ammonium per sulphate) using the Tris Base, Borate, EDTA buffer system (pH 8.3) for 2.5 h at 200V. Gels were dried and analyzed by a Cyclone Phosphorlmager system (Packard Instrument Co. Inc., Meriden, Conn.).

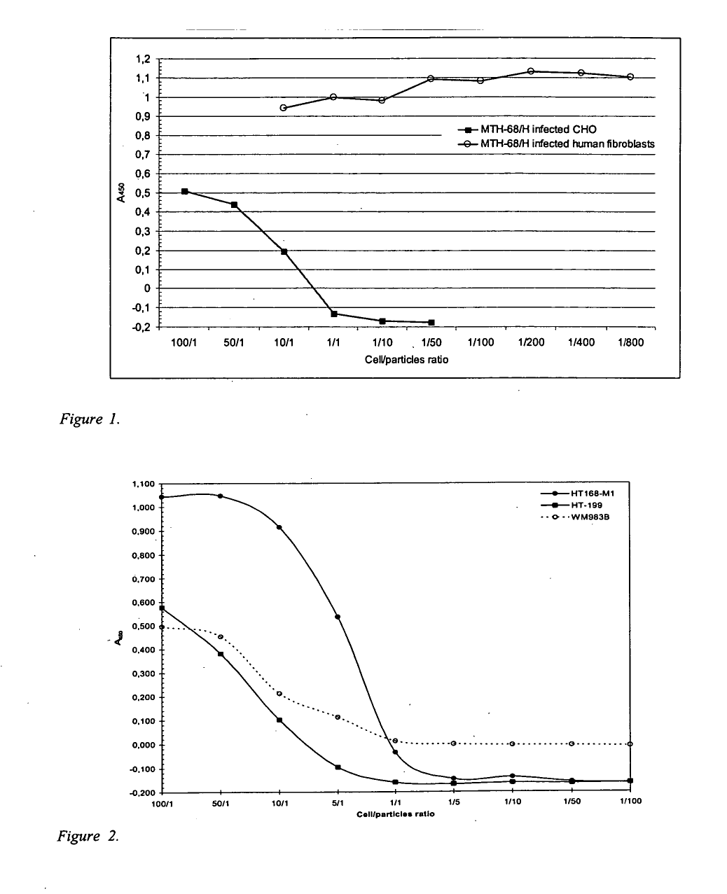

With reference to FIGS. 1-8 and Table II there can be seen the results obtained by infecting various tumor cell lines with the Herefordshire strain utilized in the form of the MTH-68/H vaccine.

WST-1 Proliferation Assays

Control and tumor cell lines were tested for MTH-68/H cytotoxicity using the WST-1 kit. The results are summarized in Table II. Human fibroblasts were completely resistant to MTH-68/H even at very high virus titers (800 particles for 1 cell, see FIG. 1). This resistance was probably not caused by the high concentration of serum (20% FBS) used to grow the cells, since the presence of serum did not inhibit the cytotoxic effect of MTH-68/H on three tumor cell lines tested (PANC-1, HeLa, MCF-7). In contrast, Chinese hamster ovary cells (CHO cell line) displayed moderate sensitivity to MTH-68/H, comparable to certain tumor cell lines (See FIG. 1 and Table II).

Melanoma Cell Lines

All three human melanoma cell lines tested (HT-199, WM983B and HT168-M1) are highly sensitive to MTH-68/H. See FIG. 2 and Table II.

Human Colorectal Cell Lines

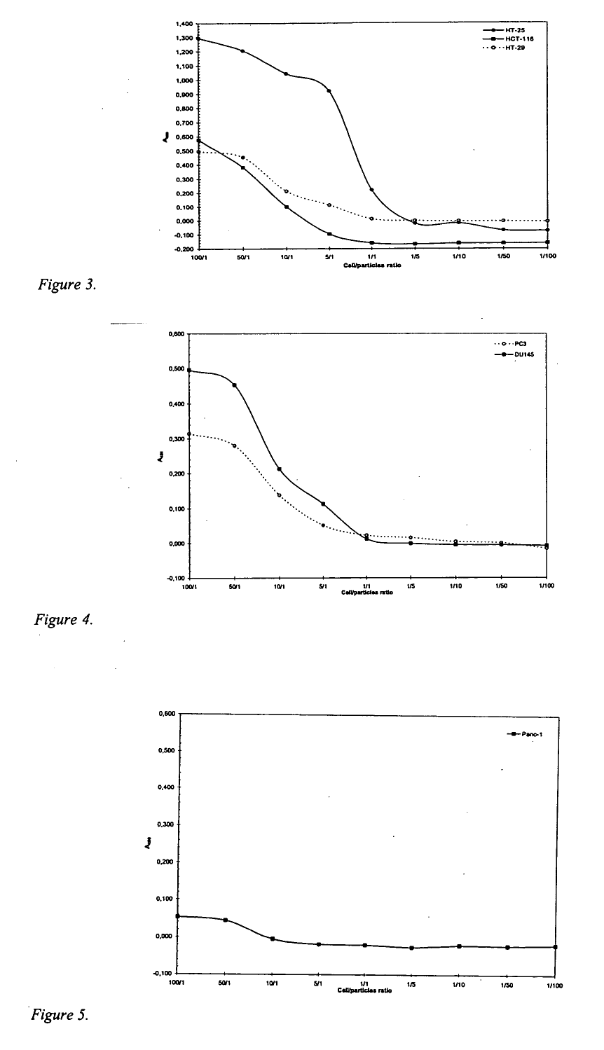

All three human colorectal cancer cell lines tested are sensitive to MTH-68/H (HT-29>HCT-116>HT-25). See FIG. 3 and Table II.

Human Prostate Cancer Cell Lines

Both cell lines tested are sensitive to MTH-68/H (PC3>DU-145). See FIG. 4 and Table II.

Human Pancreas Cancer Cell Line

The PANC-1 cell line is one of the most MTH-68/H sensitive cell lines. See FIG. 5 and Table II.

Human Large Cell Lung Cancer Cell Line

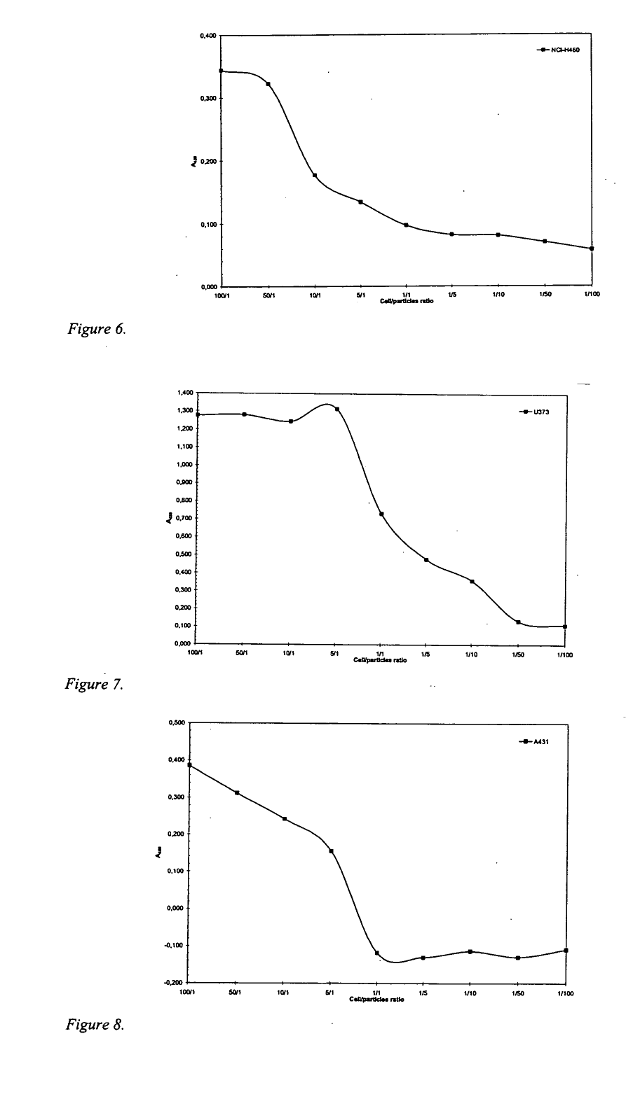

The NCI-H460 cell line is quite sensitive to MTH-68/H cytotoxicity. See FIG. 6 and Table II.

Human Astrocytoma Cell Line

U373 cells have moderate sensitivity to MTH-68/H. See FIG. 7 and Table II.

A431 Human Carcinoma Cell Line

The A431 human epithelial cancer cell line is moderately sensitive to MTH-68/H. See FIG. 8 and Table II.

To provide a basis for comparison, the NDV strains LaSota and Vitapest were also tested for their oncolytic potential. Liquid, unpurified batches of MTH-68/H, LaSota and Vitapest preparations that were isolated under identical conditions were tested on human tumor cells and compared. The preparations had the following approximate titers:

| | | |
|---|---|---|
| MTH-68/H | 10^{8.8} | particles/ml |
| LaSota | 10⁹-10¹⁰ | particles/ml |
| Vitapest | 10⁹ | particles/ml |

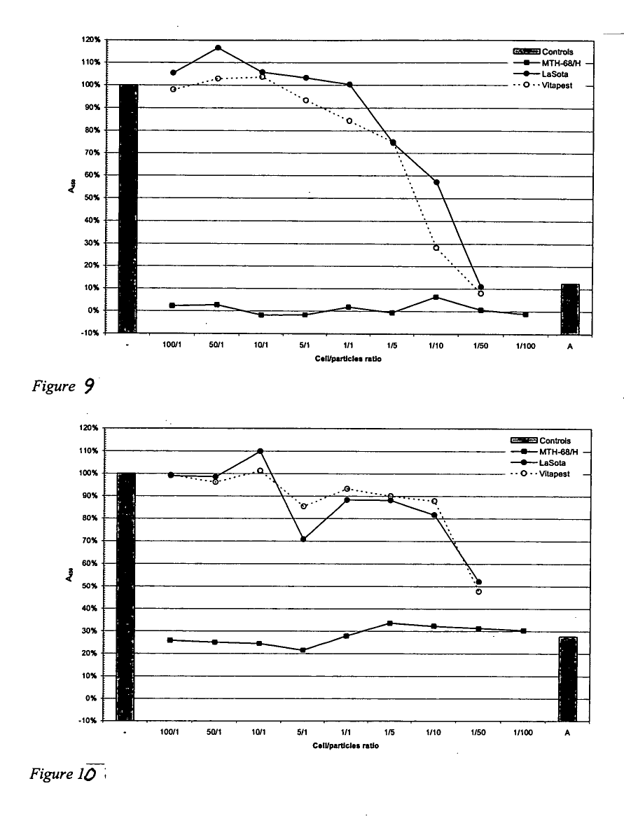

The fresh virus preparations were tested on PANC-1(see FIG. 9) and HeLa cells (see FIG. 10). On both cell lines all three NDV preparations were found to be cytotoxic, but MTH-68/H was 10³-10⁴ times more effective than LaSota or Vitapest.

TABLE II
The cytotoxicity of MTH-68/H in various cell lines

| | | | | |
|---|---|---|---|---|
| | | MTH-68/H | | |
| | | titer causing | | |
| | | 50% | Semiquantitative | |
| | | cytotoxicity* | assessment of | |
| Cell line | Source | (cell/particle) | cytotoxicity | Experiment |

Non-cancerous cell lines

| | | | | |
|---|---|---|---|---|
| Rat-1 | normal rat fibroblasts | <1/100 | − | #32 |
| NIH3T3 | normal mouse fibroblasts | <1/100 | − | #34 |
| CHO | chinese hamster ovary | 10/1-1/1 | ++ | #66, #68 |
| | human fibroblasts | <1/800 | − | #86 |

Cancer cell lines

| | | | | |
|---|---|---|---|---|
| PC12 | rat pheochromocytoma | 1/10 | + | #45 |
| HeLa | human cervical cancer | >100/1 | ++++ | #18 |
| MCF-7 | human breast cancer | 1/10 | + | #19 |
| 293T | adenovirus-transformed | >100/1 | ++++ | #20 |
| | human kidney | | | |
| Cos-7 | SV40-transformed | 1/1 | ++ | #22 |
| | monkey kidney | | | |
| PANC-1 | human pancreas cancer | >100/1 | ++++ | #80 |
| DU 145 | human prostate cancer | 5/1-1/1 | ++ | #81 |
| NC1-H460 | human large cell lung | 50/1-10/1 | +++ | #82 |
| | cancer | | | |
| HT-29 | human colorectal cancer | 10/1 | ++ | #83 |
| PC-3 | human prostate cancer | 50/1-10/1 | +++ | #84 |
| B16 | mouse melanoma | 1/10-1/50 | + | #54 #58 |
| HCT-116 | human colorectal cancer | 10/1-5/1 | ++ | #100, #105, |
| | | | | #106 |
| U373 | astrocytoma | 1/5 | + | #107 |
| HT-25 | human colorectal cancer | 5/1 | ++ | #116 |
| HT-199 | human melanoma | >10/1 | +++ | #116 |
| WM 983B | human melanoma | >10/1 | +++ | #119 |
| HT168-M1 | human melanoma | 5/1 | ++ | #119 |
| A431 | human epithelial cancer | 5/1 | ++ | #119 |

*Control: 0% cytotoxicity; anisomycin (1 μg/ml): 100% cytotoxicity.

Synergism Between MTH-68/H and Chemotherapeutics

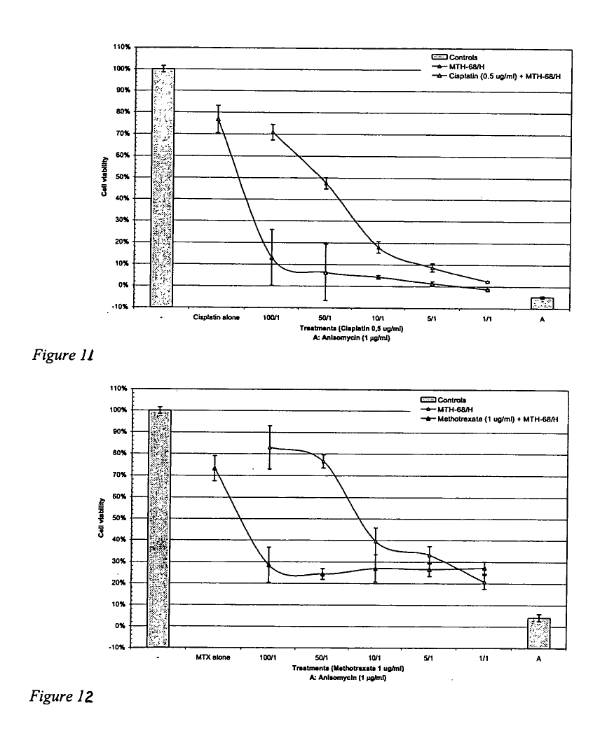

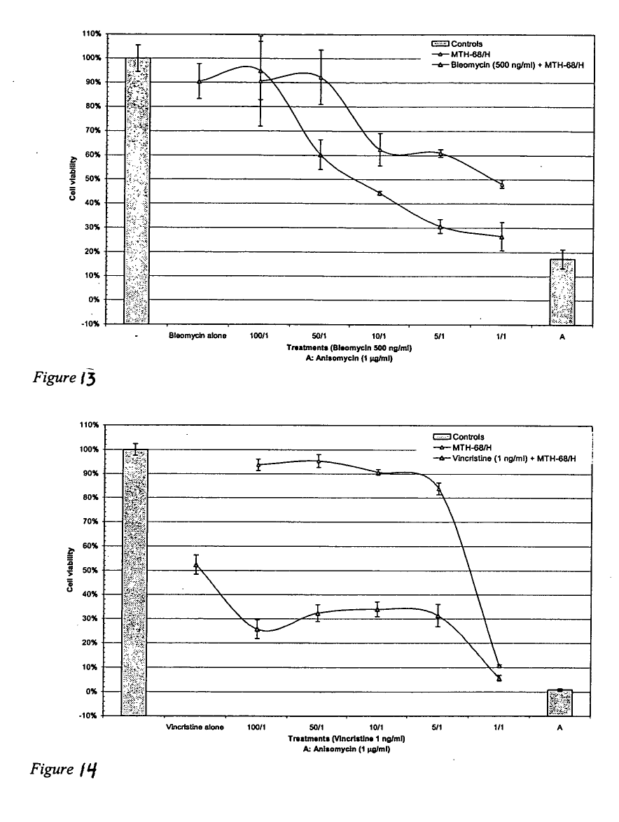

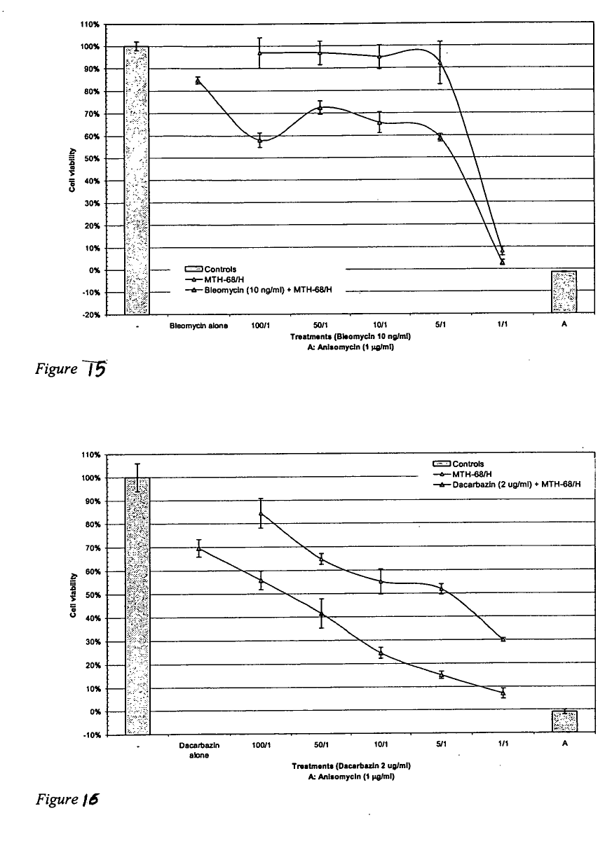

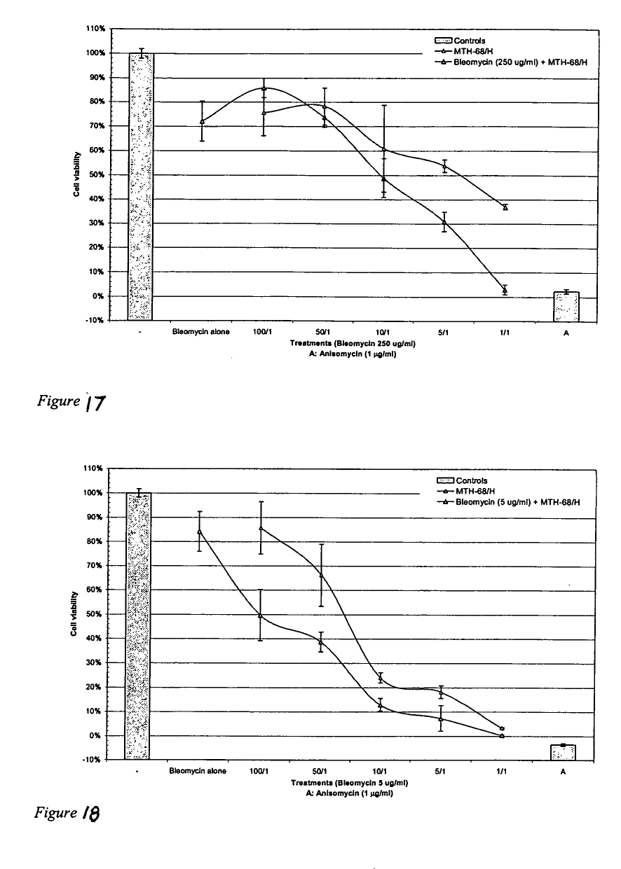

A potential clinical application of MTH-68/H is its use in combination with other therapeutic regimens, especially chemotherapeutic treatments, to increase efficacy and reduce toxicity. Therefore, several cytostatic agents were tested in combination with MTH-68/H on various tumor cell lines. The highest nontoxic concentrations of the drugs for each cell line were determined in preliminary experiments, and then these concentrations were used in combination with MTH-68/H to demonstrate synergy. The results of these tests are summarized in Table III. Graphical representations of the cytotoxicity of MTH-68/H/chemotherapeutic agent combinations on human tumor cell lines are shown in FIGS. 11-18. Each of these Figures shows the cytoxicity of the chemotherapeutic agent alone, of chemotherapeutic agent/MTH-68/H combinations in ranges from 100/1 to 1/1 and of MTH-68/H alone. In each case, it can be seen that the cytotoxicity of the combination was better than each agent alone, demonstrating the synergy of their combination.

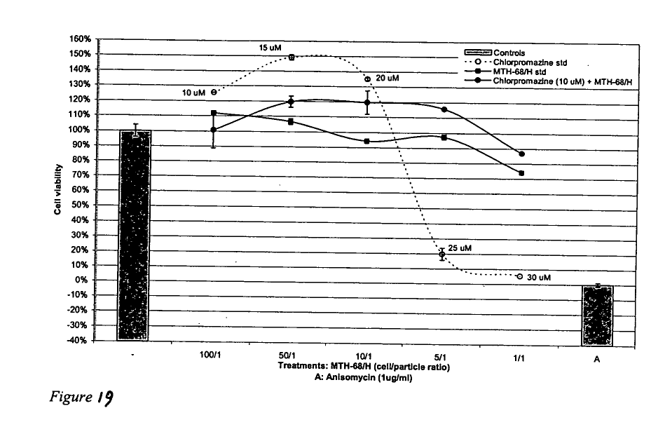

Interestingly, when similar tests were conducted using MTH-68/H and chlorpromazine on PC12, MCF-7, B16, CHO, 293T and HeLa cells, no significant synergy between chlorpromazine and MTH-68/H was observed. See Table III and FIG. 19.

While the present invention has been described in terms of specific embodiments thereof, it will be understood that no limitations are intended to the details of the disclosed methods other than as defined in the appended claims.

TABLE III
Cytotoxicity of Chemotherapeutic/MTH-68/H combinations in Various Cell Lines

| MTH-68/H + | Cisplatin | Methotrexate | Vincristine | 5-Fluorouracil | Chlorpromazine | Dacarbazine | BCNU | Bleomycin |
|---|---|---|---|---|---|---|---|---|
| PC12 | ++ | | | | − | + | | |
| | # 46 | | | | # 50 | #52 | | |
| MCF-7 | ++ | − | + | − | + | − | + | |
| | # 47 | # 47 | # 47 | #47 | # 75 | # 103 | # 103 | |
| | | | # 103 | | | | | |
| B16 | | | ++ | | − | − | | |
| | | | # 58 | | # 73 | # 54 | | |
| | | | | | # 64 | # 56 | | |
| | | | | | # 65 | | | |
| CHO | | | | | +- | | | |
| | | | | | # 66 | | | |
| | | | | | # 72 | | | |
| 293T | ++ | − | + | + | − | | − | − |
| | # 101 | # 101 | # 101 | # 101 | # 67 | | # 92 | # 93 |
| HeLa | + | | | + | − | − | − | ++ |
| | # 98 | | | # 98 | # 74 | # 125 | # 94 | # 95 |
| HCT-116 | + | | ++ | | | + | + | ++ |
| | # 105 | | # 106 | | | # 105 | # 105 | # 106 |
| Panc-1 | − | | | − | | − | − | |
| | # 125 | | | # 109 | | # 125 | # 109 | |
| HT-29 | − | − | + | − | | − | | ++ |
| | # 117 | # 122 | # 117 | # 122 | | # 122 | | # 117 |
| NCI-H460 | ++ | ++ | − | + | | − | − | ++ |
| | # 118 | # 126 | # 118 | # 126 | | # 126 | # 126 | # 126 |
| | | | # 126 | | | | | |
| PC-3 | | | | | | ++ | − | |
| | | | | | | | # 124 | |
| DU-145 | − | + | − | | | + | | − |
| | # 124 | | # 124 | | | | | # 124 |

− no synergy
+ weak synergy
++ significant synergy

1. A method for treating human tumor cells to induce apoptotic cell death thereof comprising the step of infecting the tumor cells with a combination of the Herefordshire strain of Newcastle Disease Virus and a chemotherapeutic agent.

2. A method, as claimed in claim 1, wherein the range of concentrations of chemotherapeutic agent/Herefordshire strain is in the range of 100/1 to 1/1.

3. A method, as claimed in claim 1, wherein the chemotherapeutic agent is selected from the group consisting of cisplatin, methotrexate, vincristine, bleomycin and dacarbazine.

4. A method, as claimed in claim 1, wherein the human tumor cells are selected from melanoma, colorectal, prostate, large cell lung, cervical, kidney and breast cells.
